(19) United States
(12) Patent Application Publication
(10) Pub. No.: US 2023/0054630 A1
(43) Pub. Date: Feb. 23, 2023

(54) SLEEP DIETARY SUPPLEMENT

(71) Applicant: SOCIETE DES PRODUITS NESTLE S.A., Vevey (CH)

(72) Inventors: KURT SEIDENSTICKER, Chicago, IL (US); COREY FRIESE, Chicago, IL (US); KATIE COTE, Chicago, IL (US); CRISTINA CUTRI, Chicago, IL (US)

(21) Appl. No.: 17/760,207

(22) PCT Filed: Feb. 5, 2021
(86) PCT No.: PCT/EP2021/052730

§ 371 Date: Aug. 5, 2022

Related U.S. Application Data
(60) Provisional application No. 62/971,481, filed on Feb. 7, 2020.

(51) Int. Cl. A61K 38/39; A61K 31/198; A61K 31/185; A61K 31/375; A61K 31/51; A61K 31/525; A61K 31/455; A61K 31/4415; A61K 31/519; A61K 33/06; A61K 33/30; A61K 33/04; A61K 33/00; A61K 31/405; A61K 31/197; A23L 33/28; A23L 33/175; A23L 33/16; A23L 33/155

(52) U.S. Cl. CPC A61K 38/39; A61K 31/198; A61K 31/185; A61K 31/375; A61K 31/51; A61K 31/525; A61K 31/455; A61K 31/4415; A61K 31/519; A61K 33/06; A61K 33/30; A61K 33/04; A61K 33/00; A61K 31/405; A61K 31/197; A23L 33/28; A23L 33/175; A23L 33/16; A23L 33/155; A23V 2002/00

(57) ABSTRACT

The present invention is directed to improving sleep quality and supporting recovery of joints, ligaments, and tendons with collagen peptides and dietary ingredients that support sleep quality. The supplement composition of the present invention comprises collagen, GABA, L-Theanine, 5-HTP, L-Tryptophan, and Magnesium. The recovery from exercise and physical activity is improved using this combination of components.

### BACKGROUND

Adequate and restorative sleep is vital for recovery from exercise and physical activity. Sleep deprivation can have negative effects on athletic performance including slower muscle recovery, changes in mood, increase in levels of stress hormones including cortisol, decreased glycogen synthesis, and increased ratings of perceived exertion. Increased sleep and sleep quality has been associated with improved athletic performance and recovery.

In addition to a need for healthy sleep patterns, consuming collagen peptides as a dietary supplement has been associated with enhanced recovery from exercise and provides amino acids to support muscles, joints, ligaments, and tendons that can be stressed or broken down during exercise or physical activity.

While healthy sleep can be improved via lifestyle choices such as going to sleep at the same time everyday, or making sure you get enough sleep, supplementation can play an important role and further enhance other sleep factors. These can include how long it takes to fall asleep, whether you stay asleep, or how much of that sleep occurs in the REM (Rapid Eye Movement) phase. Research has shown that several dietary supplement ingredients have inherit properties that promote these various sleep-enhancing functions, including GABA (gamma-amino-butyric acid, L-Theanine, 5-HTP, and Magnesium.

However, there remains a need to improve sleep quality and increase the uptake of collagen. The present invention resolves this deficiency and enhances the uptake of collagen peptides and improves sleep quality based on the components of the composition.

### SUMMARY

The present invention is directed to a dietary supplement composition comprising collagen peptides, GABA (gamma-aminobutyric acid), L-Theanine, 5-HTP, L-Tryptophan, and Magnesium. The composition may further include vitamins and minerals including Vitamin D3, Niacin, Vitamin B6, Folic Acid, Vitamin B12, Magnesium, Selenium, and Potassium. In one embodiment, the collagen is present in an amount of about 40 to 65 weight percent of the composition, GABA is present in an amount of about 0.2 to about 0.25 weight percent of the composition, L-Theanine is present in an amount of about 0.1 to about 0.15 weight percent of the composition, 5-HTP is present in an amount of about 0.25 to about 0.3 weight percent of the composition, L-Tryptophan is present in an amount of about 0.25 to about 0.3 weight percent of the composition, and Magnesium is present in an amount of about 1.1-1.5 weight percent of the composition. The composition of the present invention may be in the form of a powder or liquid.

The compositions of the present invention are intended to replace collagen broken down during physical activity and to support healthy sleep for optimal restoration and recovery from exercise and physical activity.

### DETAILED DESCRIPTION OF THE INVENTION

The present invention relates to a dietary supplement composition that may be consumed as a supplement to maintain collagen levels in the body, which help to support muscles, joints, tendons, and ligaments and to support healthy sleep and improve sleep quality. The compositions of the present invention comprise collagen, preferably in the form of hydrolyzed collagen peptides, in combination with GABA (gamma amino-butyric acid), L-Theanine, 5-HTP, L-Tryptophan and Magnesium. The composition may be used in the form of a liquid, solution, suspension, powder, or gel. If in powder form, the powder is mixed in a soluble liquid prior to administration as directed.

Collagen is present in the composition in an amount of about 40 to 65 weight percent of the composition, about 40 to 55 weight percent of the composition, or about 50 to 55 weight percent of the composition. Alternatively, collagen may be present in the composition in an amount of about 5-15 grams/serving, about 10-20 grams/serving, or about 10 grams/serving. In a preferred embodiment, collagen may be present in the composition in an amount of about 10 grams/serving. Collagen is a protein and may be present in any available form in the composition. However, the present invention preferably uses collagen peptides. Collagen peptides may be sourced from any known source, including but not limited to, beef, chicken, fish, eggshell membrane, and chicken egg whites.

GABA (gamma amino-butyric acid) is present in the composition in an amount of about 0.2-0.6 g/serving, about 0.3-0.5 g/serving, or about 0.35-0.4 g/serving, or about 0.4 g/serving. Alternatively, GABA may be present in the composition in an amount of about 1-3.75 weight percent, about 2.5 weight percent, about 2.1 weight percent, about 2.1-2.5 weight percent, or about 2.3 weight percent of the composition. L-Theanine is also present in the composition in an amount of about 0.1-0.3 g/serving, about 0.15-0.25 g/serving, or about 0.18-0.22 g/serving. Alternatively, L-Theanine may be present in an amount of about 0.5-2 weight percent, about 0.8-1.5 weight percent, or about 0.9-1.4 weight percent of the composition.

5-HTP is also present in the composition in an amount of about 0.02-0.08 g/serving, about 0.03-0.07 g/serving, or about 0.04-0.06 g/serving. Alternatively, 5-HTP may be present in an amount of about 0.1-0.5 weight percent, about 0.18-0.45 weight percent, or about 0.2-0.4 weight percent of the composition.

L-Tryptophan is also present in the composition in an amount of about 0.02-0.08 g/serving, about 0.03-0.07 g/serving, or about 0.04-0.06 g/serving. Alternatively, L-Tryptophan may be present in an amount of about 0.1-0.5 weight percent, about 0.18-0.45 weight percent, or about 0.2-0.4 weight percent of the composition.

Magnesium is also present in the composition in an amount of about 0.1-0.4 g/serving, about 0.15-0.3 g/serving, or about 0.18-0.24 g/serving. Alternatively, Magnesium may be present in an amount of about 0.6-0.25 weight percent, about 0.9-1.9 weight percent, or about 1.1-1.5 weight percent of the composition.

In addition, certain minerals and elements may be added to the composition as a dietary supplement. For example, selenium is appropriate for use in the composition. Selenium may be present in any suitable form, such as mustard seed extract. Selenium may be present in the composition in an amount of about 5-60 mcg/serving, 10-40 mcg/serving, 20-30 mcg/serving, or about 27.5 mcg/serving. Alternatively, selenium may be present in an amount of 0.00003-0.00038% weight percent, about 0.00006-0.00025 weight percent, or about 0.00013-0.00019 weight percent.

Vitamins may further be added to the composition. Any vitamins suitable for ingestion in a dietary supplement is suitable for use. Suitable vitamins include but are not limited to Vitamin B, including Vitamin B1 (e.g., as thiamine chloride), Vitamin B2 (e.g., as riboflavin), Vitamin B3 (e.g., niacinamide), Vitamin B6 (e.g., as pyridoxine HCl), Vitamin B5 (e.g., as pantothenic acid or D-calcium pantothenate), Vitamin B12 (cyanocobalamin), folate (e.g., as folic acid), Vitamin C (e.g., as sodium ascorbate), Vitamin D, Vitamin E, etc. In the context of the present invention, each of the vitamins may be independently present in the composition in an amount of about 0-0.2 grams/serving, about 0-1 grams/serving, about 0.2-0.8 grams/serving, 0.4-0.6 grams/serving, or about 0-0.25, about 0-0.2, about 0.001-0.2, or about 0.002-0.175 weight percent of the total composition.

Moreover, the composition may be flavored using any known natural flavors to enhance the flavor of the composition. Additionally, other additives suitable for dietary supplements, including but not limited to, coconut water powder, citric acid, malic acid, and silica.

In accordance with the present invention, suitable compositions include, but are not limited, to those described in Table 1.

TABLE 1

| | | |
|---|---|---|
| Ingredient | Amount per serving | % Daily Value |

| | | |
|---|---|---|
| Protein | 9 | 0%* |

| | | | |
|---|---|---|---|
| Vitamin D3 | 5.2 | mcg | 26% |
| Niacin (niacinamide) | 7 | mg NE | 44% |
| Vitamin B6 | 5 | mg | 294% |
| (Pyridoxine HCl) | | | |

| | | |
|---|---|---|
| Folate | 200 mcg DFE | 50% |
| | (12 mcg Folic Acid) | |

| | | | |
|---|---|---|---|
| Vitamin B12 | 2.4 | mcg | 100% |
| Magnesium (as | 230 | mg | 55% |
| magnesium lysinate | | | |
| glycinate chelate | | | |
| Selenium | 27.5 | mcg | 50% |
| Sodium | 65 | mg | 3% |
| Potassium | 55 | mg | 1% |
| Collagen Peptides | 10 | mg | ** |
| GABA (gamma amino- | 400 | mg | ** |
| butyric acid) | | | |
| L-Theanine | 200 | mg | ** |
| L-Tryptophan | 50 | mg | ** |
| Organic Mustard Seed | 11 | mg | ** |
| Extract | | | |

In an embodiment of the present invention, the following amino acid composition may be utilized:

TABLE 2

| Amino Acid | Amount per serving | |
|---|---|---|
| Alanine | 731 | mg |
| Arginine | 759 | mg |
| Aspartic Acid | 596 | mg |
| Glutamic acid | 1120 | mg |
| Glycine | 1860 | mg |
| Histidine** | 72 | mg |
| Hydroxylysine | 109 | mg |
| Hydroxyproline | 1029 | mg |
| Isoleucine**^{~} | 136 | mg |
| Leucine**^{~} | 262 | mg |
| Lysine** | 307 | mg |
| Methionine** | 54 | mg |
| Phenylalanine** | 190 | mg |
| Proline | 1038 | mg |
| Serine | 307 | mg |
| Threonine** | 172 | mg |
| Tryptophan** | 50 | mg |
| Tyrosine | 45 | mg |
| Valine**^{~} | 217 | mg |

**Essential amino acids
^{~}Branched-Chain Amino Acids (BCAAs)

In an embodiment of the present invention, the caloric intake of the supplement is less than about 100 calories, less than about 75 calories, about 50 calories, or about 45 calories per serving. Additionally, the amount of carbohydrates and total sugars in the composition is desirably low. For example, the carbohydrates may be present in an amount of less than 10 grams/serving, less than 7 grams/serving, less than 5 grams/serving, about 4 grams/serving, or about 2 grams/serving. Similarly, the total sugars is desirably kept low, e.g., less than 6 grams/serving, less than 5 grams/serving, about 2 grams/serving, or about 1 grams/serving. As noted above, the content of the protein, in the form of collagen or collagen peptides, is high, preferably above 5 grams/serving, above 7 gram/serving, or about 9 grams/serving.

### Example 1

A composition of the formula depicted in Table 1 was prepared.

1. A method for improving sleep quality comprising administering a dietary supplement to an individual in need of same comprising collagen in an amount of about 30 to 60 weight percent of the composition, glutamine in an amount of about 2 to about 3.5 weight percent of the composition, and taurine in an amount of about 0.2 to about 0.5 weight percent of the composition.

2. The method of claim 1, wherein the composition further comprises at least one branched amino acid selected from the group consisting of leucine, isoleucine, valine, lysine, phenylalanine, threonine, tryptophan, histidine, methionine, and glutamine.

3. The method of claim 1, wherein the composition is a powder.

4. The method of claim 1, wherein the composition is a liquid.

5. The method of claim 1, wherein collagen is present in an amount of about 40-55 weight percent of the composition, glutamine is present in an amount of 2.75 to about 3.0 weight percent of the composition, and taurine is present in an amount of about 0.3-0.45 weight percent of the composition.

6. The method of claim 1, wherein collagen is present in an amount of 50-55 weight percent of the composition, glutamine is present in an amount of about 2.75-3.0 weight percent of the composition, and taurine is present in an amount of about 0.4-0.42 weight percent of the composition.

7. The method of claim 1, wherein the composition comprises leucine and isoleucine.

8. The method of claim 1, wherein the composition comprises leucine, isoleucine, valine, lysine, phenylalanine, threonine, tryptophan, histidine, and methionine.

9. The method of claim 1, wherein the composition further comprises coconut water in an amount of about 10 to 20 weight percent of the composition.

10. The method of claim 1, wherein the composition further comprises coconut water in an amount of about 12 to 16 weight percent of the composition.

11. The method of claim 1, wherein the composition further comprises electrolytes selected from the group consisting of sodium, potassium, magnesium, and zinc.

12. The method of claim 2, wherein the composition comprises the nine essential amino acids in an amount of about 15-22 weight percent of the composition.

13. The method of claim 12, wherein the composition comprises the nine essential amino acids in an amount of about 8 grams per serving.

14. The method of claim 1, further comprising electrolytes selected from the group consisting of sodium, potassium, magnesium, calcium, bicarbonate, chloride, and phosphate.

15. The method of claim 1, further comprising selenium.

16. The method of claim 1, further comprising one or more of the following vitamins: Vitamin B1, Vitamin B2, Vitamin B3, Vitamin B6, Vitamin B5, folate, Vitamin C, Vitamin D, and Vitamin E.

17. The method of claim 1, wherein the composition comprises all of the following vitamins: Vitamin B1, Vitamin B2, Vitamin B3, Vitamin B6, Vitamin B5, folate, Vitamin C, and Vitamin D.

18. A dietary supplement composition comprising the following ingredients:

| Ingredient | Amount per serving | | % Daily Value |
|---|---|---|---|
| Protein | 18 | g | |
| Vitamin C | 45 | mg | 50% |
| Thiamin | 0.6 | mg | 50% |
| Riboflavin | 0.65 | mg | 50% |
| Niacin | 8 | mg NE | 50% |
| Vitamin B6 | 0.85 | mg | 50% |
| Folate | 200 | mcg DFE | 50% |
| Magnesium | 105 | mg | 25% |
| Zinc | 3.5 | mg | 32% |
| Selenium | 27.5 | mcg | 50% |
| Sodium | 300 | mg | 13% |
| Potassium | 205 | mg | 4% |
| Collagen peptides | 20 | g | |
| (from bovine) | | | |
| L-Glutamine | 970 | mg | |
| Taurine | 135 | mg | |
| Essential Acid blend: | 5 | g | |
| L-leucine, L-isoleucine, | | | |
| L-lysine, L-phenylalanine, | | | |
| L-threonine, L-tryptophan, | | | |
| L-histidine, L-methionine | | | |

19. A dietary supplement composition comprising the following ingredients:

| Ingredient | Amount per serving | | % Daily Value |
|---|---|---|---|
| Protein | 18 | g | |
| Vitamin C | 45 | mg | 50% |
| Thiamin | 0.6 | mg | 50% |
| Riboflavin | 0.65 | mg | 50% |
| Niacin | 8 | mg NE | 50% |
| Vitamin B6 | 0.85 | mg | 50% |
| Folate | 200 | mcg DFE | 50% |
| Pantothenic Acid | 2.5 | mg | 50% |
| Magnesium | 105 | mg | 25% |
| Zinc | 3.5 | mg | 32% |
| Selenium | 27.5 | mcg | 50% |
| Sodium | 230 | mg | 13% |
| Potassium | 203 | mg | 4% |
| Collagen peptides | 20 | g | |
| (from bovine) | | | |
| L-Glutamine | 970 | mg | |
| Taurine | 135 | mg | |
| Essential Acid blend: | 5 | g | |
| L-leucine, L-isoleucine, | | | |
| L-lysine, L-phenylalanine, | | | |
| L-threonine, L-tryptophan, | | | |
| L-histidine, L-methionine | | | |

20-21. (canceled)
